(19) United States
(12) Patent Application Publication
(10) Pub. No.: US 2013/0071463 A1
(43) Pub. Date: Mar. 21, 2013

(54) IMPLANTS FOR POSTOPERATIVE PAIN

(71) Applicants: Maria Palasis, Wellesley, MA (US); Upma Sharma, Somerville, MA (US); John Marini, Weymouth, MA (US); Quynh Pham, Methuen, MA (US); Toby Freyman, Waltham, MA (US); Adam Rago, Falmouth, MA (US)

(72) Inventors: Maria Palasis, Wellesley, MA (US); Upma Sharma, Somerville, MA (US); John Marini, Weymouth, MA (US); Quynh Pham, Methuen, MA (US); Toby Freyman, Waltham, MA (US); Adam Rago, Falmouth, MA (US)

(21) Appl. No.: 13/616,386

(22) Filed: Sep. 14, 2012

Related U.S. Application Data
(63) Continuation-in-part of application No. 12/620,334, filed on Nov. 17, 2009.
(60) Provisional application No. 61/535,246, filed on Sep. 15, 2011.
(60) Provisional application No. 61/598,484, filed on Feb. 14, 2012.

(51) Int. Cl. A61K 9/00; A61K 9/70; A61K 47/34

(52) U.S. Cl. CPC A61K 9/70
USPC 424/423

(57) ABSTRACT

Medical implants and methods useful in treating postoperative pain are described. The implants comprise one or more electrospun drug-loaded fibers, which fibers comprise a drug useful in the treatment of pain. The implants are implanted at sites of interest including joint capsules, bones, and subcutaneous spaces, and are secured with tissue flaps or fasteners.

### CROSS-REFERENCE TO RELATED APPLICATIONS

This application claims the benefit under 35 U.S.C. §119(e) of (i) U.S. Application Ser. No. 61/535,246 by Freyman, et al. entitled “Implants for Post-Operative Pain,” filed Sep. 15, 2011 and (ii) U.S. Application Ser. No. 61/598,484 by Sharma, et al. entitled “Acute Release of Drugs from Electrospun Implants,” filed Feb. 14, 2012 (hereinafter, “Sharma”). This application is a continuation in part of U.S. application Ser. No. 12/620,334, Publication No. 2010/0291182, by Palasis, et al. entitled “Drug-Loaded Fibers” (hereinafter, “Palasis”). The entire disclosure of each of the foregoing applications is hereby incorporated by reference for all purposes.

### TECHNICAL FIELD

The present invention relates to implants for treatment of postoperative pain.

### BACKGROUND

Postoperative pain following surgical procedures, particularly orthopedic procedures, can have a significant effect on patient recovery and quality of life, and can be difficult to treat. Oral and injectable opioids are commonly used to treat severe pain, but systemically administered opioids can be addictive, can cause adverse drug-drug interactions, and may have undesirable side effects such as respiratory depression, nausea and vomiting, somnolence, pruritis, constipation, and cognitive impairment. Additionally, patients develop tolerance to opioids, complicating treatment of pain over long periods. Local administration of pain drugs, either in solution or in delivery vectors such as liposomes, may be preferable to systemically administered drugs insofar as local administration can achieve effective drug concentrations at sites of administration while reducing systemic levels and associated side effects. However, when drugs are administered locally to surgical sites for sustained release, they may interfere with tissues or joints in a way that could cause discomfort or irritation for patients. Additionally, locally administered drugs for sustained release may migrate away from sites of post-operative pain over time. Accordingly, there is a need for drug delivery systems and methods for treating post-operative pain that are retained at surgical sites, that provide sustained release, and that minimize interference with tissues and joints and thereby minimize inflammation and patient discomfort.

### BRIEF DESCRIPTION OF THE INVENTION

The present invention addresses the need described above by providing, in one aspect, a medical implant that delivers one or more drugs for treatment of postoperative pain to a surgical site. In certain embodiments, the implant comprises one or more electrospun drug-loaded fibers having a diameter and length tailored to fit a surgical site and deliver a drug for the treatment of pain over a period of days or weeks. In certain embodiments, the implant delivers an opioid, an anesthetic, or a non-opioid analgesic. In contrast to injected drugs, liposomes or other sustained delivery vectors, implants of the present invention can be positioned within a surgical site and secured in place or otherwise resist migration, providing drug directly to a chosen area for an extended period.

In another aspect, the present invention provides methods of treating postoperative pain by placing an implant of the invention including a core-sheath fiber loaded with an analgesic within the tissue of a patient such as a joint, so that the concentration of the analgesic within the tissue increases to at least a first threshold sufficient to relieve or prevent pain over an extended period of time. In some embodiments, the concentration of the analgesic within the plasma of the patient is not increased above a second threshold at which side effects are observed. The implant can be held in place by flaps of tissue, sutures, screws, adhesive, or other fasteners. In certain embodiments, the implants are delivered to surgical sites using minimally invasive techniques.

Implants of the invention can release one or more drugs at relatively constant rates over extended periods of time. In some embodiments, a drug or drugs are released at a relatively rapid rate during an initial “burst phase” of release over approximately one day, and at a relatively slower “steady state” rate thereafter. The relative rates of release during burst and steady state phase are tuned, in certain embodiments, by applying a coating to an exterior surface of the implant or by adjusting a porosity of the implant, for example by providing a wound or coiled structure such as a yarn or a rope in which the degree of winding is selected to yield a desired porosity.

Implants of the present invention advantageously deliver analgesic drugs directly to surgical sites, achieving consistent, effective dosing locally while reducing the risk of systemic side effects. Implants of the present invention also advantageously deliver pain relieving drugs around the site of implantation over a period of days, weeks, or longer, thereby eliminating the need for repeated systemic dosing, multiple injections or implantation of transcutaneous catheters. The methods of the present invention facilitate patient ambulation and joint movement, and can contribute to improved patient outcomes, more rapid rehabilitation, shorter hospital stays and fewer readmissions due to pain.

### DESCRIPTION OF THE DRAWINGS

The figures provided herein are not necessarily drawn to scale, with emphasis being placed on illustration of the principles of the invention.

FIG. 1 is a schematic drawing of implants according to certain embodiments of the present invention.

FIG. 2 is a schematic drawing of implants secured within surgical sites according to certain embodiments of the present invention.

FIG. 3 is a schematic drawing of methods of delivering implants according to certain embodiments of the present invention.

FIG. 4 is an arthroscopic image of an implant of the present invention implanted in a joint capsule.

FIG. 5 is a photograph of an implant of the present invention implanted in the subcutaneous space outside of a joint capsule.

FIG. 6 is a series of photographs illustrating the flexibility and axial strength of an implant of the present invention.

FIG. 7 includes elution curves for ropes and/or meshes in accordance with certain embodiments of the invention.

FIG. 8 depicts the cumulative release of dexamethasone from implants of the invention having different degrees of porosity and/or rope coiling.

FIG. 9 depicts the cumulative release of dexamethasone from implants of the invention having different degrees of coiling.

FIG. 10 depicts the cumulative release of dexamethasone from implants of the invention incorporating different numbers of yarns.

FIG. 11 depicts the cumulative release of dexamethasone from implants having different degrees of yarn coiling.

FIG. 12 depicts the cumulative release of dexamethasone from implants having different degrees of drug loading.

FIG. 13 depicts the cumulative release of morphine sulphate pentahydrate from coated and uncoated ropes.

FIG. 14 depicts cumulative release of morphine sulphate pentahydrate from ropes having regions with varying degrees of winding and, consequently, porosity.

FIG. 15 depicts cumulative release in vitro of morphine sulphate from ropes of the invention.

FIG. 16 depicts cumulative release in vitro of morphine sulphate from ropes of the invention.

FIG. 17 depicts morphine levels in synovial fluid in joints containing implants of the invention.

FIG. 18 depicts cumulative release of morphine sulfate from subcutaneously implanted implants of the invention.

FIG. 19 depicts release curves of meshes of the invention.

FIG. 20 depicts release curves of meshes of the invention.

FIG. 21 depicts release curves for implants of the invention.

FIG. 22 depicts release curves for meshes of the invention in different elution media.

FIG. 23 depicts release curves for meshes of the invention including different sheath materials.

FIG. 24 depicts release curves of a mesh of the invention including fibers formed using different core polymer solvents.

### DETAILED DESCRIPTION

### Implants and Implantation Methods for Treatment of Pain

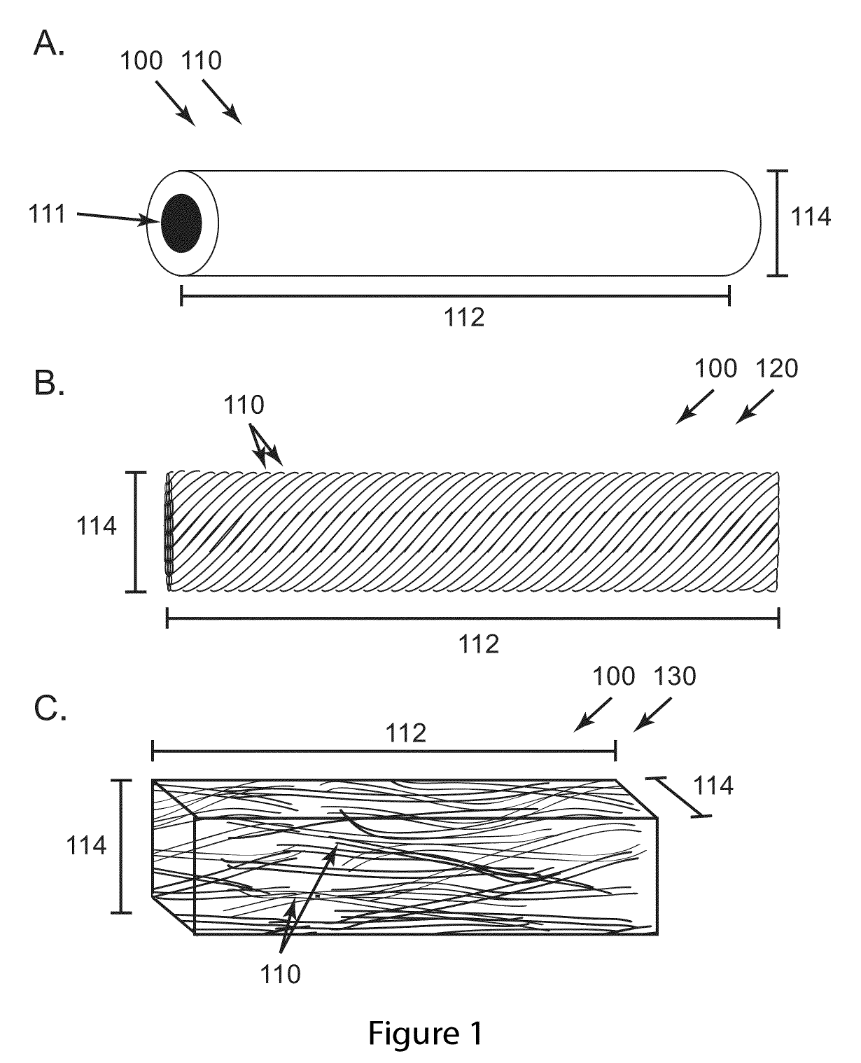

With reference to the embodiments depicted in FIGS. 1-5, implant 100 comprises at least one electrospun “core-sheath” drug-loaded fiber 110 having a drug-loaded core 111 as described in Palasis and as shown in FIG. 1A. In certain embodiments, implant 100 comprises a plurality of fibers formed into a higher-order structure such as a yarn 120, shown in FIG. 1B, or a mesh 130 shown in FIG. 1C. Though implants of the present invention comprising fibers 110 or yarns 120 are depicted in the drawings for ease of illustration, any suitable higher order structure, for example ropes, can be used. Throughout this specification, fibers and higher-order structures of the invention may be referred to by the trade name “AxioCore®” (Arsenal Medical, Inc., Watertown, Mass.).

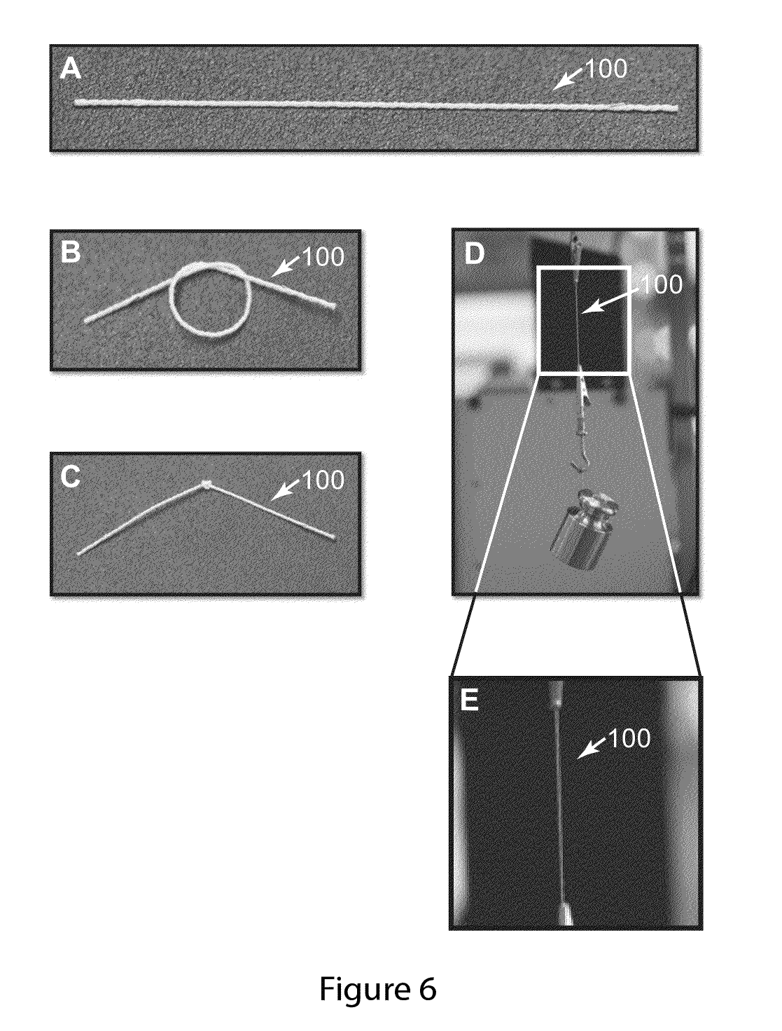

Implants of the invention are characterized by flexibility and axial strength, and can be curved or bent, inserted through tissue flaps, grasped with forceps, and tied in one or more knots without being damaged. For example, in one embodiment of the present invention as shown in FIGS. 6A, B and C, implant 100 is a 600 μm rope which is flexible enough to be looped around itself and knotted. Yet, as shown in FIGS. 6D and E, implant 100 also possesses sufficient tensile strength to support a load of 500 g. This tensile strength advantageously permits implants of the invention to be manipulated and to withstand repeated bending and pulling during and after implantation. Thus, in certain embodiments, an implant of the invention may be bent and pulled during or after implantation, for example by tissues to which they are secured, and may be used to secure multiple tissues or parts of tissues to one-another, for example as a suture or a brace. In some embodiments, the implant can be inserted through flaps of adjacent tissues, or can be wrapped and tied around adjacent tissues.

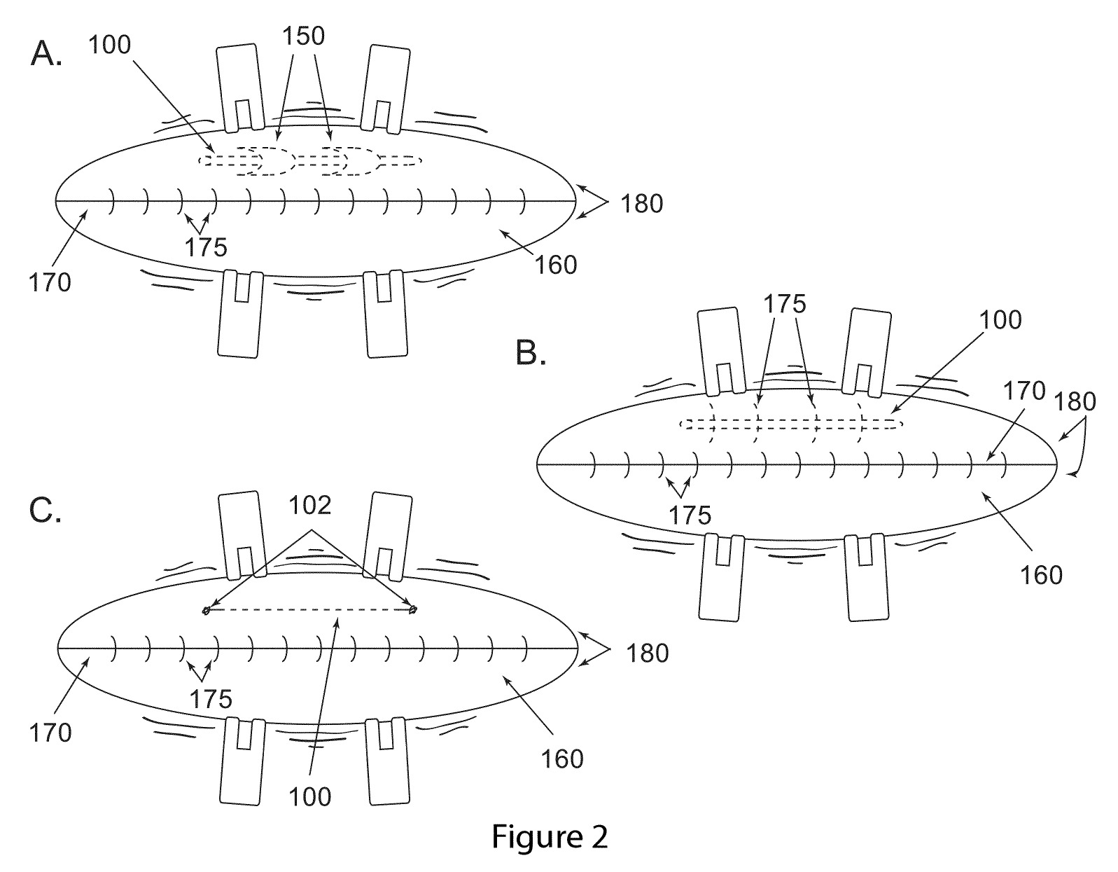

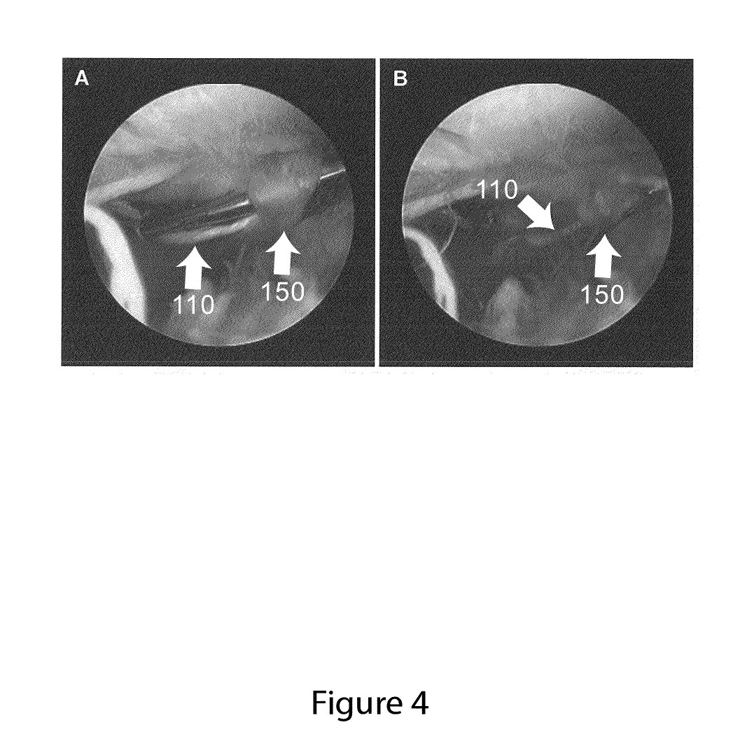

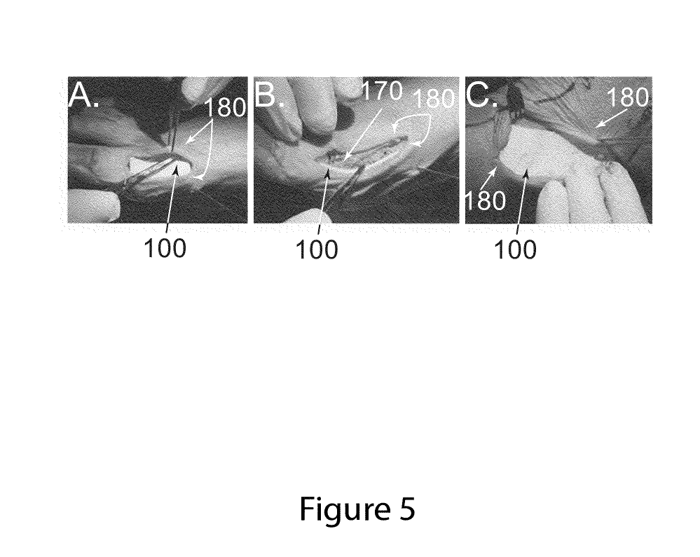

In preferred embodiments, implants of the present invention are used to relieve pain following an orthopedic medical procedure. By way of example, an implant is placed at an interior surface of a joint capsule as shown in FIGS. 2 and 4, or placed subcutaneously at a site of incision, as shown in FIG. 5. In one preferred embodiment, an implant 100 comprising one or more drug-loaded fibers 110 is implanted on the inside surface of a joint capsule 160 following an orthopedic surgical procedure and prior to closure of the surgical field. As a non-limiting example, to treat knee pain, the implant 100 is placed in one or more of the lateral and medial gutters, the superior pouch, suprapatellar space, and the posterior lateral or medial compartments. To attach the implant 100, a surgeon can pass it through the wall of the joint capsule using a needle or other device in one or more places. The implant 100 is then held in place by the resulting flap or flaps of tissue 150 or other suitable securement means. For example, FIG. 2A depicts a joint capsule 160 viewed through a retracted cutaneous incision 180; an incision 170 into the joint capsule 160 has been closed with sutures 175 following an orthopedic procedure. Implant 100 is secured within the joint capsule 160 by tissue flaps 150. Alternatively, in other embodiments such as the one shown in FIG. 2B, implant 100 is secured by sutures 175. In still other embodiments, such as the one shown in FIG. 2C, implant 100 is secured by passing its ends through the wall of the joint capsule 170 and forming a knot 102 in each end of the implant 100. The implant 100 is secured along its entire length, as shown in FIG. 2A-B, or is secured at both ends as shown in FIG. 2C, or only at one end, leaving the other end unsecured. In addition, after implant 100 is passed beneath tissue flap 150, it can be tied or sutured to the flap. In certain embodiments, implant 100 is secured using more than one means, for example sutures and insertion beneath a tissue flap.

In other embodiments, an implant is attached to a tissue such as a bone following an orthopedic procedure. The implant is secured using known fasteners including, but not limited to screws, staples, sutures or surgical adhesives. In certain embodiments, an implant is placed circumferentially around the bone and fastened at each end, for example with a suture. In other embodiments, an implant is placed within a cannulated screw after the screw has been set. In one embodiment, a mesh having dimensions of approximately 0.1 cm×2 cm×4 cm is placed along the top of the knee at the bottom of the femur following exposure of the knee joint.

In certain embodiments, implants of the present invention can be used to treat pain associated with tissue grafts. For example, in an anterior cruciate ligament (ACL) reconstruction, an implant is fastened to the graft using sutures or held in place using the securement mechanism used to hold the graft in place, for example an interference screw.

Implants of the present invention can also be placed outside of the joint capsule. In certain embodiments, as illustrated in FIG. 5, an implant 100 is positioned in the subcutaneous space above a joint capsule with forceps or any other suitable positioning tool known in the art, then the cutaneous incision is closed. As discussed above, the implant 100 is secured using sutures, adhesives, or other means known in the art. In other embodiments, an implant of the present invention is attached to a bone outside of a joint capsule, using screws, sutures, adhesives, or other means known in the art.

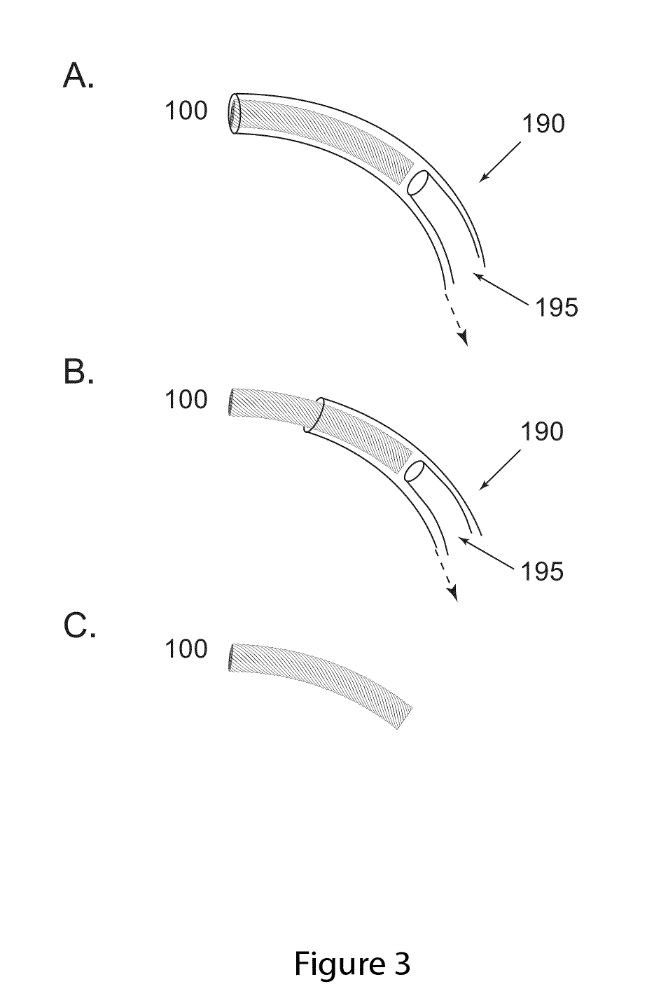

The cutaneous incision 180 and the incision into the joint capsule 170 closed with sutures 175 as depicted in FIGS. 2 and 5 are characteristic of embodiments in which the implant is delivered via open surgery. It will be evident to those skilled in the art, however, that implants of the present invention can be delivered to a patient by any suitable means known in the art, including minimally invasive means such as arthroscopy or through catheters. For example, in certain embodiments, such as the one depicted in FIG. 3, an implant 100 is carried within the lumen of a catheter 190 to a desired position. In certain embodiments, the catheter 190 includes an internal guidewire or pushrod 195 within its lumen to facilitate steering of the catheter 190, and to permit the catheter 190 to be retracted over the implant 100, discharging the implant 100 as depicted in FIGS. 3B and C. In other embodiments, the implant is held in a pair of forceps and inserted through a tissue flap or flaps. In other embodiments, a needle is used to insert the implant through a tissue flap. In still other embodiments, the implant is delivered using a specialized device that holds the implant in a set of jaws and forms tissue flaps using a blunt end.

Implants of the present invention are well suited to control pain resulting from procedures involving osteotomies, or which result in bone damage. Certain preferred indications for the use of implants of the present invention afford access to the inside of a joint capsule and are associated with significant postoperative pain. Examples of such procedures are total knee replacements, total hip replacements, total shoulder replacements, partial replacement of the knee, hip or shoulder, arthroscopic or open ACL repairs, bunionectomies, hallux valgus surgery, hammertoe surgery, ankle fusion or replacement, spinal fusion, and iliac crest bone harvest.

Implants of the present invention can be sized to fit a particular implantation site. As shown in FIG. 1, Implant 110 is characterized by a length 112 and at least one width or diameter 114, which dimensions vary depending on the intended use of the implant. Implant 110 preferably has a diameter 114 of 50 to 5000 microns and more preferably 500 to 2000 microns. The length 112 is preferably 0.5 to 10 cm and more preferably 1 to 5 cm, although the appropriate length will be determined by the size of the joint being treated, the severity of expected pain, and the therapeutic agent selected. In some embodiments, the implant is supplied in a standard length and physicians or other end users may cut the implant to a desired length prior to implantation. As non-limiting examples, an implant of approximately 1 centimeter in length is preferred for use in a bunionectomy, while an implant length of 5 centimeters of more is preferable in a total knee replacement. In preferred embodiments, the implant is fully elongated or nearly so when implanted. In certain alternate embodiments, however, the implant may be positioned in any suitable configuration, for example curved, doubled over, coiled or wadded. In other embodiments, the implant 100 includes fibers 110 delivered to a surgical site in suspension, as described in United States Publication No. 2010/0291182 by Palasis, et al. entitled “Drug-Loaded Fibers, the entire disclosure of which is incorporated herein by reference.”

The fiber or fibers 110 of implant 100 are loaded with a drug suitable for the treatment of pain. In preferred embodiments, the fiber or fibers 110 are loaded with an opioid such as morphine sulfate, morphine base, codeine, hydrocodone, hydromorphone, methadone, meperidine, butorphanol, buprenorphine, nalbuphine, alfentanil, sufentanil, fentanyl, tramadol, pentazocine, propoxyphene, oxycodone, thebaine, diacetylmorphine, oxymorphone, nicomorphine, remifentanyl, carfentanyl, ohmefentanyl, ketobemidone, dextropropoxyphene, etorphine, nalbufine, levorphanol, or tramdol. In preferred embodiments, the fiber or fibers release the opioid into the surrounding tissue and fluid for a period of 1 to 45 days. More preferably, drug release continues for between 3 and 14 days. In preferred embodiments, the fiber or fibers 110 comprise bioresorbable polymers that are resorbed on timescales longer than 1 to 45 days, permitting the rate of drug elution from fiber or fibers 110 to be controlled separately from the rate of fiber degradation. Longer resorption timescales also improve tolerability and biocompatibility by reducing inflammation associated with resorption. Alternatively, shorter resorption timescales can be used to partially control the rate of drug release—i.e. the rate of release will be a function of the rate of resorption.

The fiber or fibers preferably release drugs such as morphine at a rate of 0.005 to 10 mg/day, more preferably at a rate of 1 to 5 mg/day. In alternate embodiments, the fiber or fibers release buprenorphine. Buprenorphine is used as an analgesic for the treatment of moderate to severe post-operative pain, and may be superior to morphine for certain applications due to its higher potency, which may achieve effective pain control at lower drug volumes, permitting implant size to be decreased and thereby decreasing the amount of polymer that must be used and resorbed. Additionally, buprenorphine is a mixed agonist and antagonist of different opioid receptors, and may have a superior profile for side effects such as respiratory depression. Buprenorphine is preferably released from implants of the invention at a rate of 10-1200 micrograms/day, more preferably at a rate of 400-1000 micrograms/day. In other alternate embodiments, the fiber or fibers release hydromorphone or another morphine derivative. In still other embodiments, the fiber or fibers contain a potent lipophilic opioid, preferably fentanyl or sufentanil. If the implant contains sufentanil, the drug is preferably released at a rate of 5 to 10 micrograms/day.

In other embodiments, the fiber or fibers contain a local anesthetic, including as non-limiting examples, bupivacaine, lidocaine, chloroprocaine, cinchocaine, etidocaine, levobupivacaine, mepivacaine, ropivacaine or tetracaine. In still other embodiments, the fiber or fibers contain another class of drug that is useful in the treatment of pain, including, without limitation, a GABA receptor antagonist, barbiturate, alpha-2 adrenergic receptor agonist, COX-2 inhibitor, serotonin-noradrenaline reuptake inhibitor, amphetamine, vanilloid receptor antagonist, non-steroidal anti-inflammatory, acetylcholine receptor agonist, somatostatin analog, calcium channel blocker, sodium channel blocker, potassium channel blocker or chloride channel blocker. Specific drugs that can be used in certain embodiments of the present invention include, without limitation, baclofen, butalbitol, clonidine, rofecoxib, celecoxib, dexmedetomidine, gabapentin, ibuprofen, ketamine (S-, R-, or racemic mixture of enantiomers), ketorolac, midazolam, neostigmine, octreotide, somatostatin, saxitoxin, or ziconotide.

### Control of Drug Release Kinetics

While the foregoing disclosure focuses on the use of core-sheath fibers, homogenous electrospun drug-loaded fibers as described in Palasis and Sharma can also be used in implants of the invention. Homogeneous electrospun fibers typically release drugs very rapidly (up to 90% release, by mass, within 24 hours) when exposed to a water-containing environment, a phenomenon termed “burst release” to distinguish it from the sustained “steady-state” kinetics also observed in implants of the invention. Burst release is also observed in core-sheath fibers, and in higher order structures such as yarns, ropes, tubes and meshes, whether those structures include homogeneous fibers or core-sheath fibers. The amount of burst release and/or steady-state release can be varied in implants of the invention according to the methods that follow.

Without wishing to be bound to any theory, it is thought that the amount of burst release (amount of drug released in 1 day) in higher order structures (such as ropes, yarns, and meshes) varies with the degree of accessibility of individual fiber surfaces to water, i.e. with the porosity of the structure: the higher the porosity of the structure, the more rapid the release of drug therefrom. The porosity (Φ) of a patch, yarn, rope or other structure is the fraction of the bulk volume (V) of the structure that is not occupied by fibers, (V_{f}), and can be estimated according to formula (I) below:

$$Φ = \frac{V - {V}_{f}}{V} \qquad ( 1 )$$

As the degree of coiling of a structure increases (i.e. as the structure is coiled more tightly) the bulk volume of the structure decreases to approach the volume of the fibers comprising it (i.e. the porosity of the structure decreases), decreasing the accessibility of water to fiber surfaces internal to the structure.

The inventors believe that, when homogeneous drug-loaded fibers are formed into yarns or ropes, the release of drug therefrom can be controlled by varying the porosity of such structures, which in turn may be controlled by varying parameters including, but not limited to, (1) the extent of twisting of individual fibers as they are formed into yarns (“yarn coiling”); (2) the extent of twisting of yarns as they are formed into ropes (“rope coiling”); (3) the number and thickness of the yarns used to form ropes; and (4) the homogeneity or heterogeneity of diameters among fibers used to form yarns, or among the yarns used in ropes. The degree of yarn coiling can be controlled by varying, among other things, the rate of twisting of individual fibers as they are collected and the duration of the collection period, both as described in Palasis. The release of drug can be further tuned by forming implants that include features affecting porosity with other features, such as coatings or enclosures, or by varying the hydrophobicity of the materials used to form fibers and implants of the invention.

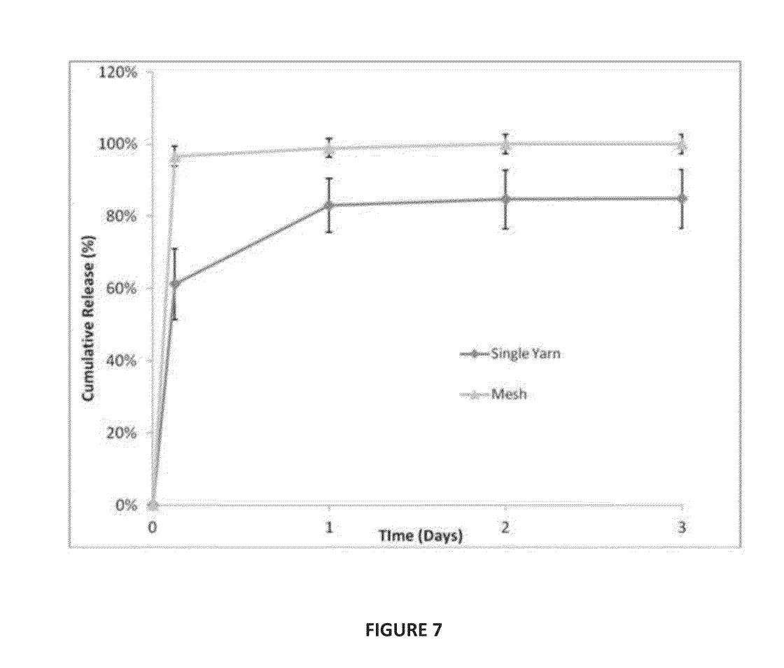

Burst release of drugs such as morphine sulfate pentahydrate can be assayed by immersing drug-loaded fiber devices in PBS. At specified timepoints, the PBS bath is changed and morphine sulfate levels measured, for example by reversed-phase high-performance liquid chromatographic method (RP-HPLC) or by ultraviolet-visible (UV-Vis) spectroscopy. FIG. 7 depicts drug release from fibers of 80:20 75/25 L-PLGA (poly (lactic-co-glycolic acid):morphine sulfate pentahydrate in the geometry of either meshes or yarns. Yarns were collected for one minute on collectors rotating at 85 RPM while meshes were collected on a mandrel as hollow tubes. The magnitude of burst release of drug from relatively more porous electrospun meshes (n=4, porosity >80%) is substantially greater than release from relatively less porous yarns (n=4, porosity ˜40%).

Similar experiments exploring the relationship between implant porosity and drug elution were performed with structures made of fibers consisting of 70:30 85/15 L-PLGA:dexamethasone. In one experiment, drug elution was measured over 35 days for the ropes listed in Table 1, below:

TABLE 1
Collection Conditions for Samples Shown in FIG. 7:

| | | Yarn Collection | | |
|---|---|---|---|---|
| Sample: | Collector RPM | Time (seconds) | Rope Revolutions | Φ |
| 126-78-5 | 30 | 70 | 40 | 5-8% |
| 126-87-6 | 30 | 70 | 3 | 25% |
| 126-93-3 | 30 | 70 | 2 | 34% |

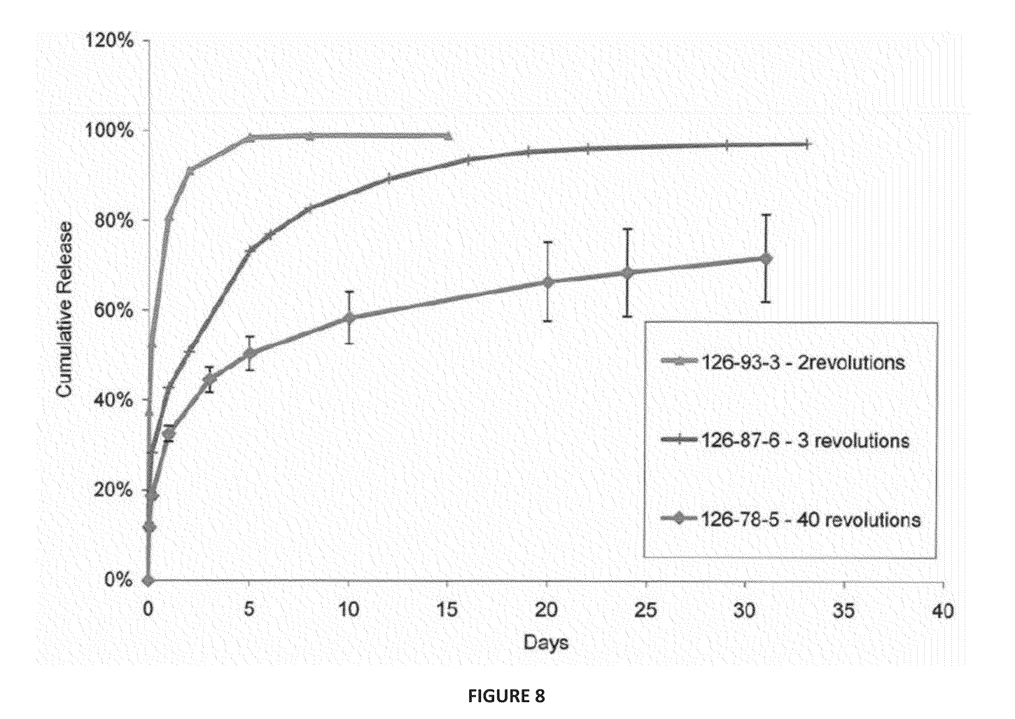

FIG. 8 shows results from three rope samples made of yarns formed under identical conditions and having roughly the same degree of yarn coiling. However, due to differing extents of rope coiling, the porosity of the ropes varied from approximately 5% up to 34%, and the cumulative release of dexamethasone from the ropes varied with their porosity. In sample 126-93-3, which had a calculated porosity of 34%, 80% of the dexamethasone content of the rope had been released by day 1, and 100% had been released by day 5. In sample 126-187-6, having a porosity of 25%, 80% release was achieved by day 7, and 100% release was achieved after approximately 35 days. Finally, in the lowest porosity (−5%) sample 126-78-5, only approximately 60% of the dexamethasone content was released within 30 days.

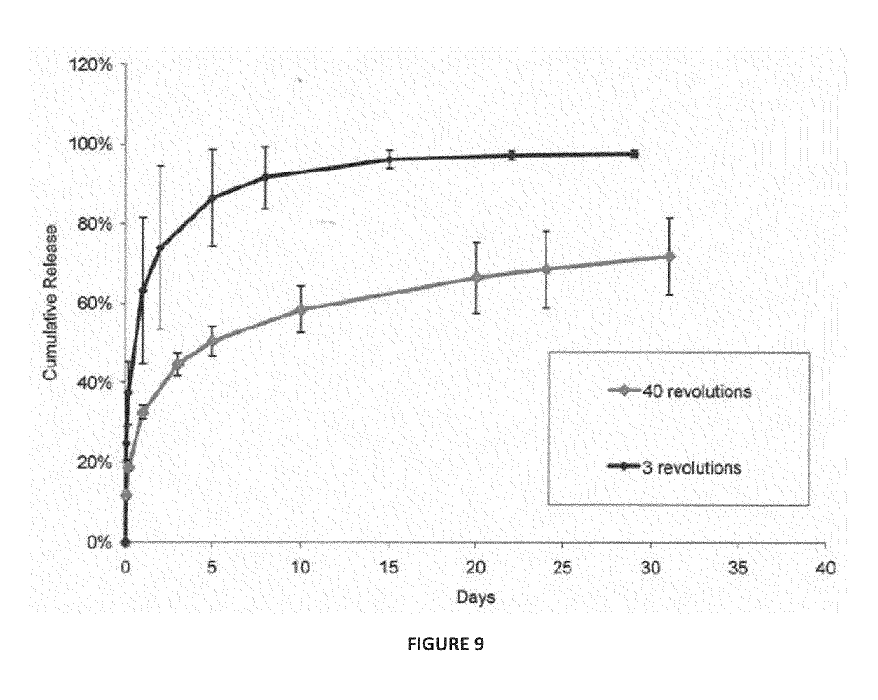

FIG. 9 illustrates that the porosity of a structure also affects the variability of drug release therefrom. Drug elution was measured from the dexamethasone-containing ropes listed in Table 2, below, which had undergone either 3 or 40 rope revolutions:

TABLE 2
Rope Coiling and Porosity of Samples Shown in FIG. 9:

| | | Number of |
|---|---|---|
| Sample | Φ | revolutions |
| Sample 1 | 8% | 40 |
| Sample 2 | 8% | 40 |
| Sample 3 | 5% | 40 |
| Sample 4 | 25% | 3 |
| Sample 5 | 31% | 3 |
| Sample 6 | 35% | 3 |

As is shown in the figure, the release of dexamethasone from samples having undergone 3 rope revolutions was quite variable, though all 3-revolution ropes had released nearly all of their dexamethasone content by day 15. By contrast, the variability of release from 40-revolution ropes was relatively small over the first 20 days of measurement, and became more variable thereafter. Error bars represent standard deviation.

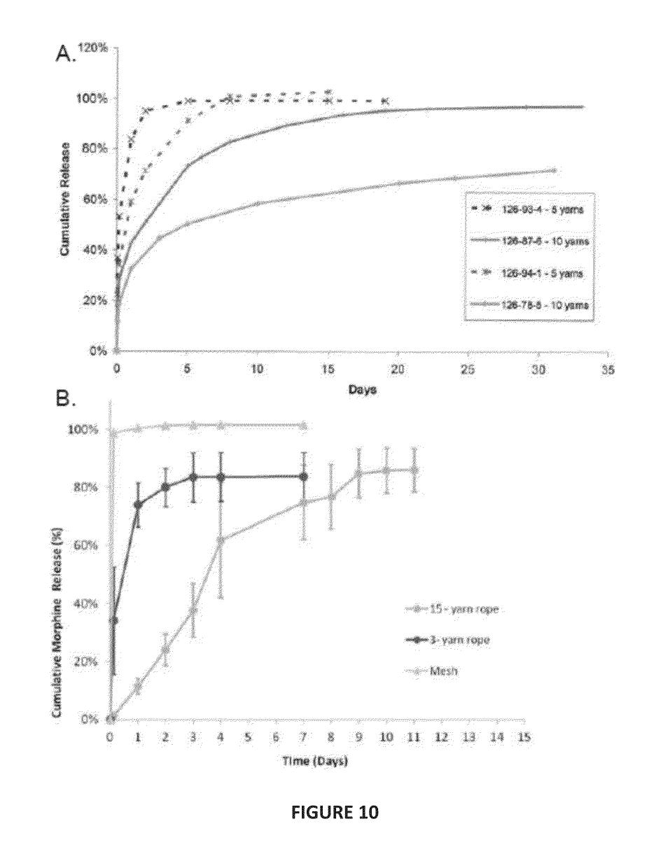

Apart from porosity, the number of yarns comprising a rope also has a strong effect on the rate of drug elution therefrom, as shown in FIGS. 10A and B. Table 3A, below, shows the ropes used in the experiment summarized in FIG. 10A:

TABLE 3A
Rope Coiling and Numbers of Yarns
Comprising the Samples Shown in FIG. 10A:

| | Rope | | Rope Thickness | |
|---|---|---|---|---|
| Sample: | Revolutions | Number of Yarns | (μm) | Φ |
| 126-78-5 | 40 | 10 | 360 | 5-8% |
| 126-94-1 | 40 | 5 | 250 | 6% |
| 126-87-6 | 3 | 10 | 510 | 25% |
| 126-93-4 | 3 | 5 | 288 | 25% |

In general, as is evident in FIG. 10, ropes comprising relatively fewer yarns release drug more rapidly than ropes comprising relatively more yarns having similar porosity, and, when yarn number is kept constant, ropes having relatively higher porosity release drug more rapidly than ropes having relatively lower porosity. While the inventors do not wish to be bound to any particular theory, it is thought, when yarn thicknesses are kept roughly constant, ropes having fewer yarns are not as thick as ropes having more yarns, and by extension the relative surface area—and the relative accessibility of fiber surfaces to water—of ropes with fewer yarns is higher per unit mass of rope than ropes having more yarns.

The effects of yarn number and porosity on drug release are also illustrated in FIG. 10B for rope implants containing morphine sulfate pentahydrate and 75/25 L-PLGA comprising either 3 or 15 yarns. The rope implants used in the experiment are summarized in Table 3B, below:

TABLE 3B
Numbers of Yarns
Comprising the Samples Shown in FIG. 4B:

| | | Rope Thickness | |
|---|---|---|---|
| Sample: | Number of Yarns | (μm) | Φ |
| 3-Yarn Rope | 3 | 530 | 40% |
| 15-Yarn Rope | 15 | 13540 | 31% |

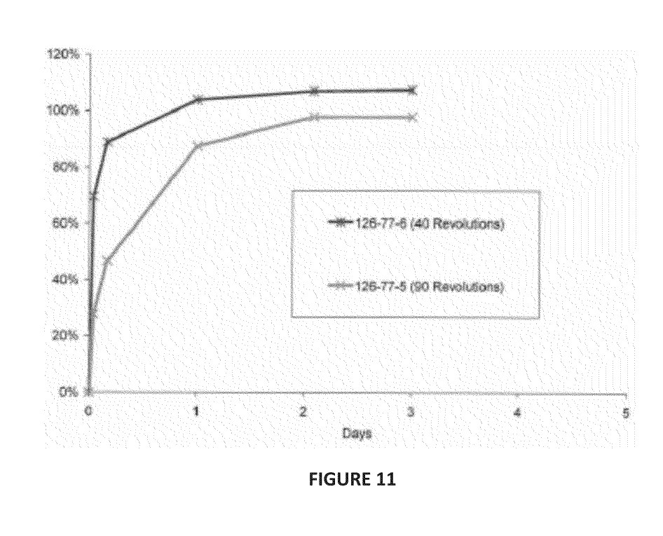

FIG. 11 shows the effect of the extent of yarn coiling on dexamethasone drug elution from single yarns. The yarns used in the experiment were formed using substantially identical fabrication conditions differing only in that, in sample 126-77-6, the collected yarn underwent 40 revolutions while in sample 126-77-5 the collected yarn used underwent 90 revolutions. In the sample with 40-revolutions, the dexamethasone was fully released after approximately one day, while in the sample with the 90-revolution yarns the dexamethasone was only ˜80% released at the same interval.

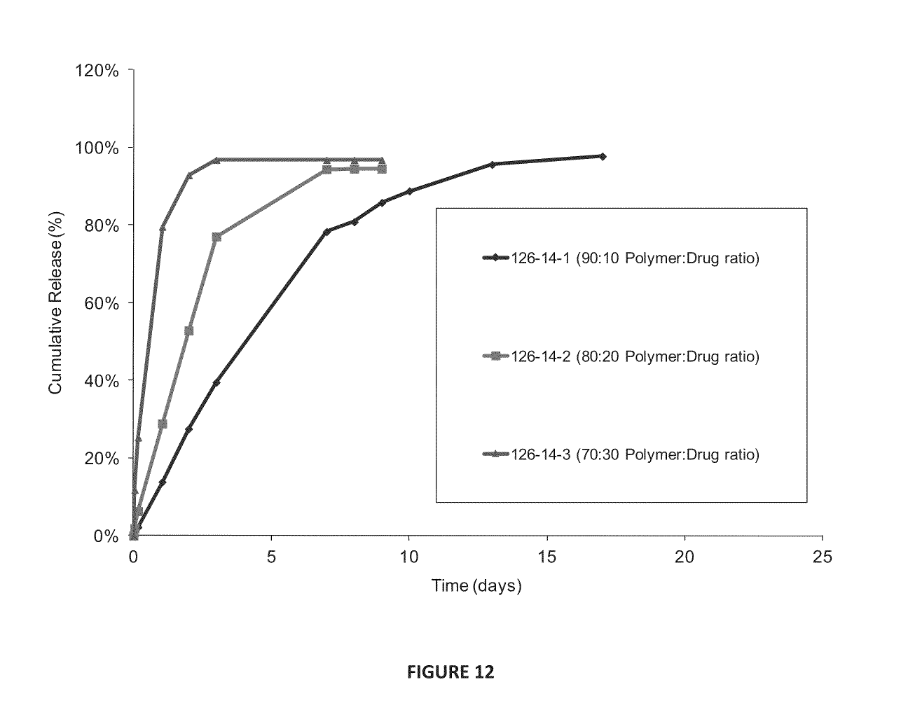

The inventors have also discovered that the rate of burst release in higher-order structures can be tailored by varying the composition of the fibers within such structures. FIG. 12 illustrates the effect of varying the polymer:drug ratio of fibers on drug release from ropes. Table 4 lists the samples used in the experiment:

TABLE 4
Fiber Composition of Samples Shown in FIG. 12:

| | Polymer:Drug | Rope Thickness | |
|---|---|---|---|
| Sample: | Ratio | (μm) | Φ |
| 126-14-1 | 90:10 | 212 | 3% |
| 126-14-2 | 80:20 | 242 | 7% |
| 126-14-3 | 70:30 | 242 | 9% |

In general, as FIG. 12 illustrates, as more drug is incorporated into fibers, burst release increases.

Burst release kinetics of yarns and ropes may be further modified by varying the degree of tension or compression applied to fibers or yarns during the twisting process: though not wishing to be bound to any theory, it is thought that as the tension applied to individual fibers or yarns increases during twisting, the fibers will tend to lie more closely together, reducing the porosity of the finished structure. Similarly, burst release kinetics may be modified by varying the direction of twisting of yarns and ropes: rope twisting may be in the direction opposite of yarn twisting (e.g. a rope with a left hand twist comprising yarns with a right hand twist), as is typical, or in the same direction (e.g. a rope with a right-hand twist comprising yarns with right-hand twist). Again, without wishing to be bound to any theory, it is believed that when yarn twisting and rope twisting directions are the same, fibers within the structure will line up more closely, leaving less room for water to access fiber surfaces and slowing burst release, while more space will exist between fibers in ropes in which the directions of yarn- and rope-twisting are opposite, resulting in better access and greater burst release.

Though the embodiments discussed above focus on ropes, the principles disclosed herein are broadly applicable to structures incorporating drug-loaded fibers. Drug release from patches, tubes and other structures comprising multiple drug-loaded fibers, as described in Palasis, may be tailored to specific applications by modulating the porosity of these structures, for example by forming them under compression or vacuum, to minimize spaces between fibers. Such structures may also be folded, crushed, crumpled, etc. to reduce porosity. Meshes and portions of meshes may also be stretched and twisted to tailor porosity and drug release. As discussed above, though not wishing to be bound to any theory, stretching results in closer alignment of fibers, permitting closer packing and decreasing porosity. In some embodiments, mesh strips may be twisted to form yarn-like structures and, optionally, woven or bound together to form superstructures having different porosity relative to the meshes used as starting materials. In some embodiments, a yarn or rope may be enclosed by a mesh.

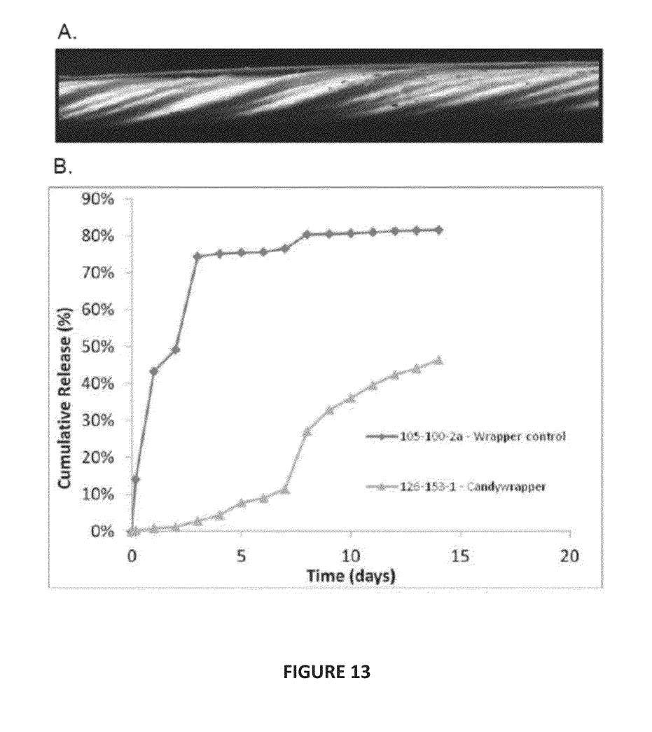

In preferred embodiments, implants are coated with polymeric coatings such as hydrogels—as discussed in Palasis—or nonpolymeric coatings such as wax, which coatings may dissolve or erode away. Such coatings may advantageously alter the burst release characteristics of an implant, as well as improving the resistance of yarns and ropes to unraveling. The coatings may be applied as heat-shrink tubing, sprayed on, dipped, or applied in any other suitable way known in the art. This is illustrated in FIG. 13, in which a 15-yarn, 75/25 L-PLGA rope device containing morphine sulfate pentahydrate is placed within a hollow polymer tube. The polymer tube was fabricated via dip-coating a mandrel into 75/25 L-PLGA polymer solution and allowing the solvent to evaporate, leaving behind a thin hollow tube of polymer. The polymer tube was removed from the mandrel and the rope device then placed inside. The tubing/rope composite was then subjected to heat whereby the polymer tube was stretched to conform as close as possible over the rope device. The ends of the polymer tube were sealed via solvent melding. See FIG. 13A. As shown in FIG. 13B, encapsulating the implant significantly reduces the extent of burst release (compare 126-153-1 Candywrapper sample to 105-100-2a Wrapper control).

In some embodiments, coatings are applied to implants, i.e. completed ropes, meshes or yarns, or to components, such as fibers or yarns that will subsequently be assembled into higher-order structures. Multiple coatings may be applied, for example first to implant components such as fibers or yarns, and again to the assembled implant. Alternatively, multiple coatings may be applied only to the exterior of the implant, or to different portions of the implant.

The coatings are preferably biocompatible, and may be bioabsorbable and/or mechanically or chemically erodible. Coatings may optionally contain drugs, such as antibiotics, antimycotics, anticoagulants, etc., and may be porous, or solid, and may be permeable, semipermeable or impermeable.

Implants of the invention may include multiple regions of different porosities or even porosity gradients. In some embodiments, yarns and ropes may be formed having regions of varying porosity by varying the extent of twisting among these regions. In some embodiments, these regions may be separated by pinch points, at which they are compressed and secured during the twisting process. These pinch points may optionally be delineated by any suitable means known in the art, including the inclusion of radiopaque, fluorescent, or pigmented marker bands as is described in Palasis.

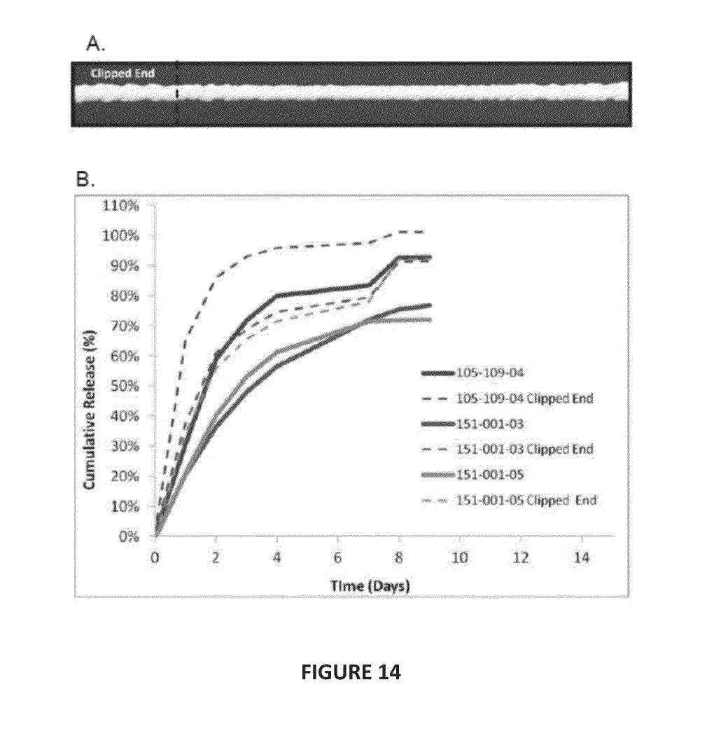

Ropes and yarns having varying porosity may be fabricated by varying the degree of twisting among regions during rope or yarn formation, for example by pinching off regions of the rope at different stages of the twisting process. As shown in FIG. 14A,varying the degree of twisting along the length of a rope results in varying thicknesses as well and, when burst release among less tightly wound regions (“clipped end”) and more tightly wound regions is compared, the less tightly wound regions demonstrate a higher degree of burst release as shown in FIG. 14A. In other embodiments, rope or yarn twisting is varied by welding (e.g. by exposure to a solvent for the polymer) ropes or yarns having different degrees of twisting to one-another. Ropes and yarns may be welded to one another end-to-end or alongside one another. In some embodiments, an implant may be formed from different ropes or yarns, for example exhibiting differing degrees of twisting, stretching, made from different materials, etc., that are optionally connected to one another or contained within a single coating.

In some embodiments, the ends of yarns, ropes and patches may be fixed by heat-setting, partial melting, chemical finishing, or any other suitable means known in the art, to prevent unraveling of the structures during their residence in a body. In addition, the surface of the fiber may be modified to reduce porosity. For example, this can be accomplished by brief exposure to heat. Thus, increasing the temperature on the surface sufficiently high to melt fibers together, but not allowing sufficient heat transfer to melt fibers on the interior. Alternately, brief exposure to a solvent for the polymer fiber (e.g. solvent vapor) can be used to similar effect.

Implants having porosity gradients as described above may be implanted individually to provide varying release rates from different portions of the implant. For example, one portion of the implant can be relatively more porous (or can lack a coating, etc.), and can release drug in a burst, while another portion of the implant that is relatively less porous (or which incorporates a coating, etc.) provides more steady-state drug release. Alternatively, such implants may be cut or otherwise separated into separate pieces, thereby forming smaller implants having relatively uniform drug release properties. One or more of these smaller implants may then be implanted into a patient in order to tailor administration of the drug. For example, an implant having a porosity gradient can be cut into a fairly porous implant and a relatively less porous implant, both of which can be implanted into a patient. In this system, the more porous implant provides relatively rapid, burst-like drug release, while the less porous implant provides sustained release. The manner in which the larger implant with the porosity gradient is cut into smaller pieces can be selected by a physician or an end user based upon the burst and/or steady-state release kinetics desired, as well as the amount of drug desired to be released into the patient. The amount of drug to be released into the patient can be determined, in turn, by the weight of the patient or other dosing guideline.

The principles of the invention are further illustrated by the following non-limiting examples:

### Example 1

### AC33 and AC34 Yarns and Ropes for Sustained Release of Morphine Sulphate Pentahydrate

Morphine eluting implants were fabricated through a coaxial electrospinning process as described in Palasis utilizing a core and sheath needle (20 and 10 gauge respectively). The core solution contained a 12% weight 75:25 PLGA polymer with respect to an acetonitrile solvent. Morphine sulfate was added to the core solution at 40% weight with respect to the polymer and mixed with a high-shear centrifugal mixer for 1 minute at 2000 rpm. For AC33, the core and sheath needles extruded solution at 2 and 3 mL/hr respectively. For AC34, the core and sheath needles extruded solution at 0.8 and 3.5 mL/hr respectively. The sheath solution for both devices was an 8% weight 75:25 PLGA polymer with respect to a 1:1 (by vol) tetrahydrofuran/dimethylformamide (THF/DMF) solvent. Extruded solutions were electrospun onto two ground collectors spaced approximately 10 centimeters apart for one minute to create one yarn. This process was repeated 15 times to create additional yarns. The yarns were dried for two days at 60° C. and then twisted around one another 8 times to create a rope with a calculated porosity of approximately 27%. The devices were dried for an additional hour at 60° C. to allow the polymer to set. Each rope was trimmed to approximately 4 cm in length and 1.2 mm and contained less than 250 ppm of residual DMF solvent. AC33 and AC34 contained approximately 11.4 mg (23 wt %) and 3.8 mg (13 wt %) of morphine, respectively.

### Example 2

### AC54 Yarns and Ropes for Sustained Release of Morphine Sulphate Pentahydrate

Implants were fabricated through an electrospinning process in which drug loaded polymer fibers are collected and twisted around one another between a small gap in a 20% relative humidity atmosphere. The core solution contained a 12% weight 75:25 PLGA polymer with respect to an acetonitrile solvent. Morphine sulfate was added to the core solution at 40% weight with respect to the polymer and mixed with a high-shear centrifugal mixer for 1 minute at 2000 rpm. The sheath solution consisted of a 14.7 wt % blend of 50:50 DL-PLGA and 75:25 PLGA polymer (1:1 by mass) dissolved in a 1:1 (by vol) THF:DMF solvent system. Sheath and core solution were delivered from their respective nozzles at flow rates of 3 and 2 ml/h, respectively. Upon electric field activation, the solutions were electrospun onto two grounded collectors spaced approximately 10 centimeters apart for one minute to create one yarn. This process was repeated 15 times to create additional yarns. The fifteen yarns were dried for three days at 60° C. and then twisted around one another 8 times to create a rope with a porosity of approximately 24%. The devices were dried for an additional hour at 60° C. to allow the polymer to set. The final individual rope was approximately 4 cm in length and 1.2 mm in diameter and contained 17% weight morphine (approximately 7.5 mg) and less than 250 ppm of residual DMF solvent.

### Example 3

### In Vitro Performance of Ropes of the invention

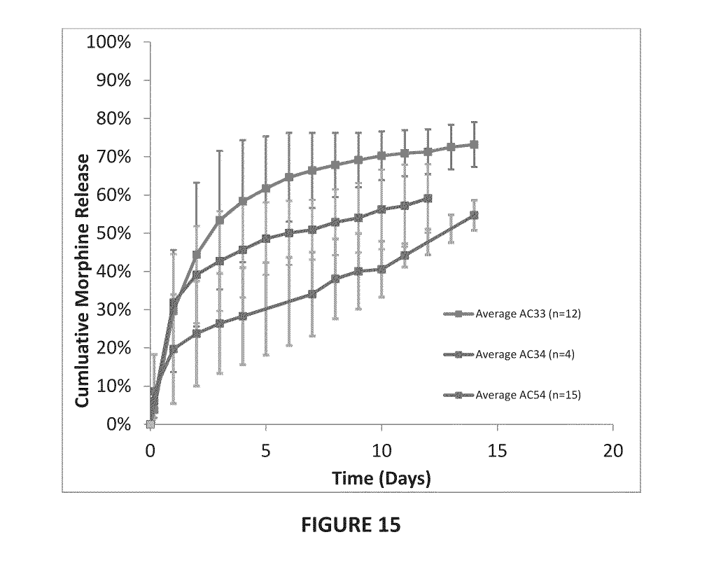

Morphine sulfate levels were measured during in vitro elution in PBS by using a reversed-phase high-performance liquid chromatographic method (RP-HPLC), and the cumulative release curves for AC33, AC34, and AC554 are shown in FIG. 15. The method utilizes a reverse phase C18 column (Symmetry C18, 5.0 um, 4.6x150 mm, Waters, Milford, Mass., USA). The HPLC system consists of a Waters Breeze Separator system with a 1525 isocratic pump, column heater, 2487 Dual Wavelength Absorbance Detector and a 717Plus Auto sampler. The mobile phase for isocratic elution consisted of a mixture of 610/375/15 v/v/v of water, acetonitrile, acetic acid with 80 mM ammonium acetic and 5 mM SDS. Under the optimum separation conditions, morphine eluted at 3.2 min. Detection was at 240 nm and 50 uL of sample was injected each time.

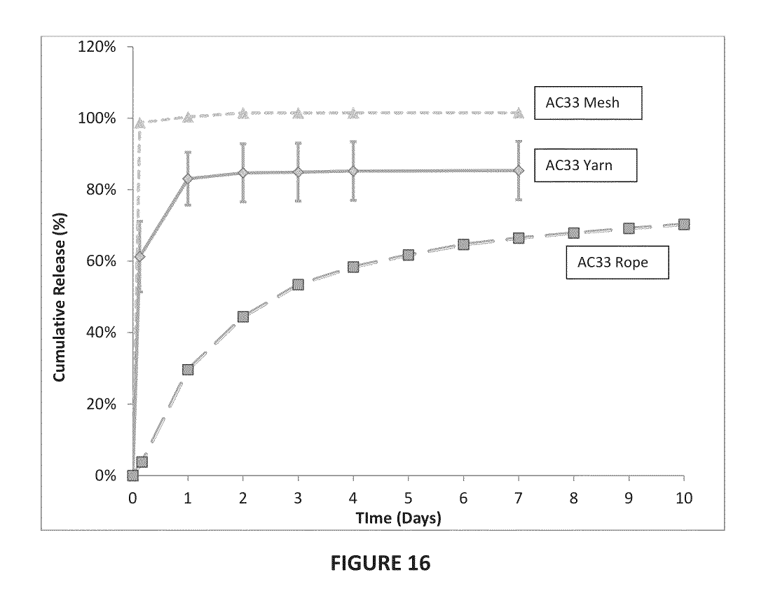

The release of morphine sulfate from AC33 ropes is specific to the way in which it was fabricated. A comparison of the release of AC33 ropes vs AC33 yarns or meshes (FIG. 16) illustrates that drug is rapidly released from mesh structures, while sustained release is achieved to a limited degree by yarns and to a greater degree by ropes. As discussed above, the release rate of drug from implants of the invention is impacted by the higher-order structure of the implant. AC33 fibers have a diameter of 800 nm, which is less than half of the size of the morphine sulfate particles produced by the high shear mixing process (˜2 microns), and it is believed that fiber sheaths may not fully encapsulate the particulate cores. Without wishing to be bound to theory, it is believed that, when meshes comprising AC33 fibers are placed in elution media, morphine sulfate particles are immediately exposed and thus diffuse rapidly, resulting in burst release. However, in yarns and ropes, coiling of adjacent fibers is believed to result in the enveloping of at least some of these fibers, resulting in less rapid release. In other formulations, such as the ACMMS formulations described in Example which have a diameter larger than the morphine sulfate particles, the release from meshes may not be as rapid as the release from AC33 meshes.

### Example 4

### In Vivo Performance of Ropes: Intra-Articular Implantation

To characterize drug concentrations achieved in vivo by devices of the invention, intra-articular implantation of ropes of the invention was performed in sheep knees. The sheep model was selected specifically for these studies because the knee anatomy of the sheep is most similar in size and tissue physiology to humans than other species. (Martini L, Fini M, Giavaresi G, Giardino R. Sheep model in orthopedic research: a literature review, Comp Med. 2001 August; 51(4):292-9)

Devices were implanted for 3 and 7 days, then retrieved (“explanted”). Each animal received two implants: an AC33 device in one knee, and an AC34 device in another knee. [CORRECT?] All implantation and explantation procedures were performed by direct visualization of the intra-articular space. Implants were implanted beneath the synovial membrane on the lateral side of the femur. A stainless steel pushrod was inserted into the membrane to create space for the delivery system and device. The implant was then advanced into the joint, under direct visualization. To deploy the device, the pushrod (placed against the implant inside the catheter) was held in place while the catheter was withdrawn, as shown in FIG. 3. This left the implant in the joint but allowed removal of the catheter. To secure each implant, a single suture was placed at the exposed end of implant. Tissue adhesive and sutures were used to close the synovial membrane. During explant, all devices were located easily by the surgeon. All devices were discovered during explant while attached to the required suture. All devices were explanted in one piece.

During the period of implantation, synovial fluid samples from each knee and plasma samples were collected at regular intervals and analyzed by a tandem mass spectrometry scope with a liquid chromatography method (Agilux, Worcester, Mass.). The samples collected were shipped with dry ice and stored at −80° C. prior to analysis. A solid phase extraction with an Oasis MCX plate (Waters, Milford, Mass., USA) was used to clean up the synovial and plasma samples. The analysis was carried out with an ACE C18-AR (2.1×50 mm id, 3 μm particle size). The mobile phase for morphine analysis consisted of acetonitrile and 2 mM ammonium acetate, acetonitrile and 0.1% pentafluoropropionic acid in 0.1% formic acid in water for vitamin B6 analysis. The analytes were detected on a triple quadrupole mass spectrometer (API 4000, Sciex, ON, Canada) equipped with an electrospray ionization source operating in the positive ion mode. Quantification was performed using the selective reaction monitoring (SRM) mode to study precursor→product ion transitions for morphine (m/z 286.19→152.2).

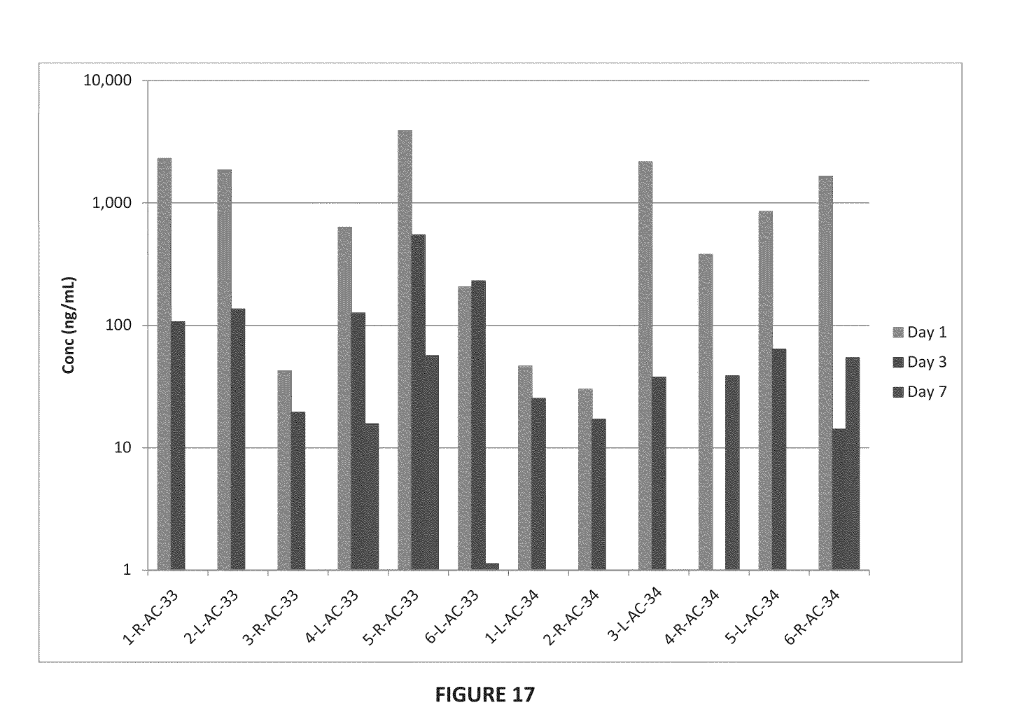

Morphine concentrations were determined for 29 of 30 successful taps. All synovial fluid taps for AC33 devices registered quantifiable levels of morphine across the seven day study, including devices that were determined to be outside the synovial membrane. One synovial fluid tap from the six AC34 devices registered a value below quantifiable limits. All remaining synovial fluid taps for AC34 devices registered quantifiable levels of morphine across the seven day study, including devices that were determined to be outside the synovial membrane. Morphine tap concentrations are shown in Table 5. FIG. 17 depicts morphine levels in the synovial fluid for all samples tested; the results demonstrate sustained release from ropes of the invention over several days.

TABLE 5
Synovial fluid tap concentration.
“BQL” indicates that the morphine concentration was
below quantifiable limits.

| | | Day 1 | Day 3 | Day 7 |
|---|---|---|---|---|
| | | Tap-Morph | Tap-Morph | Tap-Morph |
| Formulation | Subject | (ng/mL) | (ng/mL) | (ng/mL) |
| AC-33 | 1-R | 2310 | 107 | — |
| | 2-L | 1870 | 136 | — |
| | 3-R* | 43 | 20 | — |
| | 4-L* | 635 | 126 | 16 |
| | 5-R | 3900 | 549 | 57 |
| | 6-L | 206 | 231 | 1 |
| | Average | 2072 | 256 | 29 |
| | Std Dev | 1519 | 203 | 40 |
| AC-34 | 1-L* | 47 | 25 | — |
| | 2-R* | 30 | 17 | — |
| | 3-L | 2170 | 38 | — |
| | 4-R | 380 | NA** | 39 |
| | 5-L | 853 | 64 | BQL |
| | 6-R | 1660 | 14 | 55 |
| | Average | 1266 | 39 | 31 |
| | Std Dev | 802 | 25 | 28 |

*Devices outside of synovial space, not included in averages
**Tap volume below 0.1 mL, sample could not be analyzed

Residual morphine levels in devices explanted on days 3 and 7 are shown in Table 6. AC33 morphine sulfate values dropped from 12% to 8% between days 3 and 7 when compared to their predicted loading. AC34 morphine sulfate values dropped from 15% to 10% between days 3 and 7 when compared to their predicted loading. Four devices that were not located within the intra-articular space were not included in the averages.

TABLE 6
Morphine extraction from explanted devices

| | | | Morphine Remaining vs. | | |
|---|---|---|---|---|---|
| | | | Predicted Loading (%) | | |
| Formulation | Day | Animal | Value | Average | Std Dev |
| AC-33 | 3 | 1-R | 12% | 12% | 1% |
| | | 2-L | 11% | | |
| | | 3-R* | 10% | | |
| | 7 | 4-L* | 9% | 8% | 2% |
| | | 5-R | 9% | | |
| | | 6-L | 6% | | |
| AC-34 | 3 | 1-L* | 26% | 15% | — |
| | | 2-R* | 14% | | |
| | | 3-L | 15% | | |
| | 7 | 4-R | 8% | 10% | 2% |
| | | 5-L | 11% | | |
| | | 6-R | 10% | | |

*Devices outside of synovial space, not included in averages

Morphine concentrations in plasma were also measured at 1 and 4 hours in addition to days 1, 3, and 7. The morphine sulfate concentration for days 1, 3, and 7 were below quantifiable levels. The 1 and 4 hour concentration levels are shown in Table 7. Each animal had one AC33 and AC34 AxioCore device implant, 2 devices total.

TABLE 7
Morphine concentration in plasma (ng/mL)

| | Timepoint | | | | | |
|---|---|---|---|---|---|---|
| Sheep | 0 | 1 Hour | 4 Hour | Day 1 | Day 3 | Day 7 |
| 1 | BQL | 4.0 | 11.3 | BQL | BQL | BQL |
| 2 | BQL | 6.5 | 5.4 | BQL | BQL | BQL |
| 3 | BQL | 3.8 | 6.9 | BQL | BQL | BQL |
| 4 | BQL | 6.2 | 7.9 | BQL | BQL | BQL |
| 5 | BQL | 5.3 | 9.8 | BQL | BQL | BQL |
| 6 | BQL | 6.8 | 9.5 | BQL | BQL | BQL |
| Average | — | 5.4 | 8.5 | — | — | — |
| Std Dev | — | 1.3 | 2.2 | — | — | — |

### Example 5

### In Vivo Performance of Ropes: Subcutaneous Implantation

To characterize drug release from devices of the invention, AC54 devices were implanted and explanted subcutaneously in a rabbit model. The rabbit SQ model was selected as a standard method for testing of in vivo drug elution. Each animal received two implants, one in each of the left and right flanks. The animals were sacrificed per schedule at day one, three, and seven post implantation (N=3 per timepoint). There were no device related deaths or adverse events. Animal health remained normal throughout the duration of the study as measured twice daily by MPI staff veterinarians. Animals were observed for clinical signs of test article effect and body weights were measured Morphine levels in plasma were low after the first day of implant. Device implant location and surgical procedure revealed no gross adverse inflammation or effects during the study as visually documented in the images.

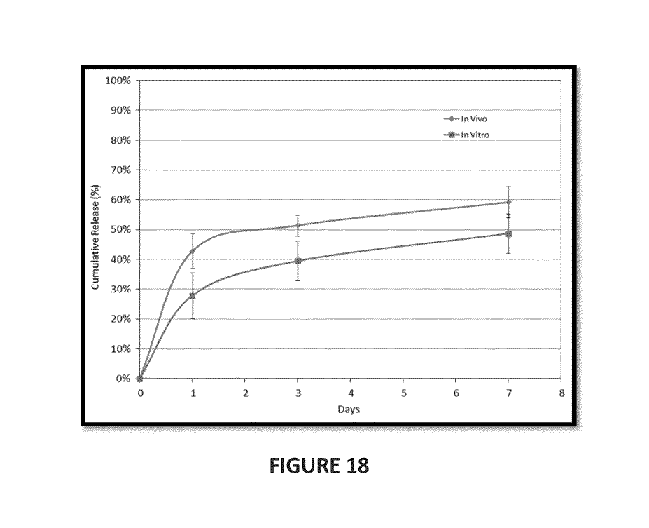

Upon explant, the drug remaining in each device was measured and compared with the predicted implant loading. The six subcutaneous devices for each timepoint had an average of 57±6%, 49±4%, and 41±5% morphine sulfate remaining in the devices with respect to days 1, 3, and 7 when compared to its predicted loading. Morphine extraction values are outlined in Table 8 and FIG. 18. AC54 devices fabricated for this study averaged 164 ug of morphine sulfate per milligram of device post fabrication.

TABLE 8
Morphine extraction from explanted devices

| | | | Cumulative | | Cumulative | |
|---|---|---|---|---|---|---|
| Explant | | | Released | Remaining | Released | Remaining |
| Day | Subject | Device | (μg/mg) | (μg/mg) | (%) | (%) |
| 1 | 501 | 145-178-6A | 55 | 110 | 33% | 67% |
| | | 145-178-7B | 74 | 91 | 45% | 55% |
| | 502 | 145-178-4B | 82 | 83 | 50% | 50% |
| | | 145-178-9B | 64 | 101 | 39% | 61% |
| | 503 | 145-178-1B | 77 | 88 | 47% | 53% |
| | | 145-170-2A | 72 | 93 | 44% | 56% |
| | Average | | 71 | 94 | 43% | 57% |
| | Std Dev | | 10 | 10 | 6% | 6% |
| 3 | 504 | 145-178-2B | 91 | 74 | 55% | 45% |
| | | 145-175-6A | 82 | 83 | 49% | 51% |
| | 505 | 145-178-3B | 92 | 73 | 56% | 44% |
| | | 145-171-2A | 86 | 79 | 52% | 48% |
| | 506 | 145-174-4A | 80 | 85 | 49% | 51% |
| | | 145-164-3A | 78 | 87 | 47% | 53% |
| | Average | | 85 | 80 | 51% | 49% |
| | Std Dev | | 6 | 6 | 4% | 4% |
| 7 | 507 | 145-177-4A | 95 | 70 | 57% | 43% |
| | | 145-175-5B | 101 | 64 | 61% | 39% |
| | 508 | 145-177-3B | 114 | 51 | 69% | 31% |
| | | 145-175-3A | 89 | 76 | 54% | 46% |
| | 509 | 145-172-3A | 97 | 68 | 59% | 41% |
| | | 145-173-2B | 92 | 73 | 55% | 45% |

| | | | | |
|---|---|---|---|---|
| Average | 98 | 67 | 59% | 41% |
| Std Dev | 9 | 9 | 5% | 5% |

Comparisons of morphine release in in vitro and in vivo are set out in Tables 9 and 10. Results suggest the drug elution from the device in vivo is more rapid than expected from in vitro results during the first day of release. The cumulative release curves from days 2 through 7 for both profiles are comparable.

TABLE 9
Morphine cumulative release % In Vivo/In Vitro

| | Day 1 | | Day 3 | | Day 7 | |
|---|---|---|---|---|---|---|
| Condition | Release | Std Dev | Release | Std Dev | Release | Std Dev |
| In Vivo | 43% | 6% | 51% | 4% | 59% | 5% |
| In Vitro | 28% | 8% | 39% | 7% | 49% | 7% |

TABLE 10
Morphine cumulative release values In Vivo/In Vitro

| | Day 1 | | Day 3 | | Day 7 | |
|---|---|---|---|---|---|---|
| | Cumula- | | Cumula- | | Cumula- | |
| | tive Re- | Std | tive Re- | Std | tive Re- | Std |
| Con- | leased | Dev | leased | Dev | leased | Dev |
| dition | (μg/mg) | (μg/mg) | (μg/mg) | (μg/mg) | (μg/mg) | (μg/mg) |
| In | 71 | 10 | 85 | 6 | 98 | 9 |
| Vivo | | | | | | |
| In | 46 | 15 | 65 | 10 | 79 | 12 |
| Vitro | | | | | | |

### Example 6

### Meshes for Sustained Release of Morphine Sulphate Pentahydrate

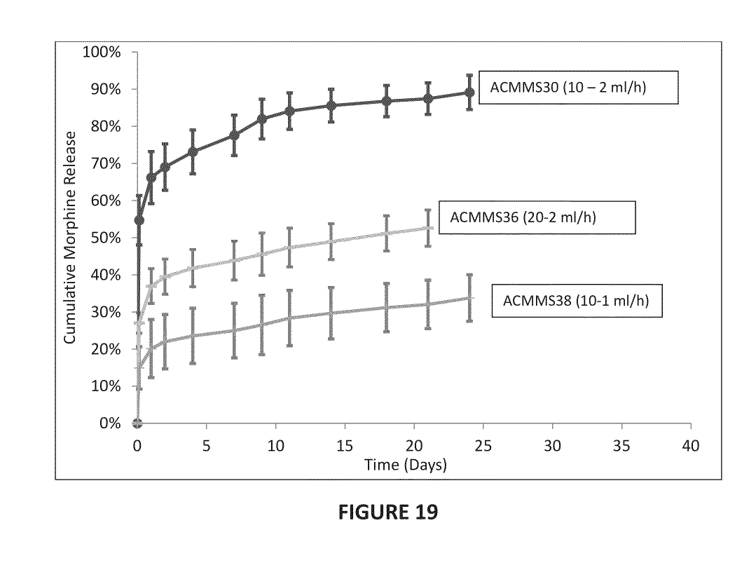

Sustained release of morphine sulfate was also achieved via encapsulation techniques in a mesh form factor. FIG. 19 depicts several sustained release formulations with different levels of burst and duration of release. Coaxial electrospinning using distinct sheath and core solutions was used to fabricate meshes according to these embodiments. The sheath solution was comprised of a 3.5 wt % 85/15 L-PLGA in 6:1 (by vol) chloroform:methanol solution. The core solution was comprised of a 15 wt % PCL in 6:1 (by vol) chloroform:methanol solution containing 20% morphine sulfate pentahydrate relative to the PCL.

In order to demonstrate control of release, different sheath and core flow rates were used: ACMMS30 had sheath and core flow rates of 10 and 2 ml/h, respectively; ACMMS36 had sheath and core flow rates of 20 and 2 ml/h, respectively; and ACMMS38 had sheath and core flow rates of 10 and 1 ml/h, respectively. Fibers were collected onto a grounded rotating mandrel located ˜20-30 cm away, resulting in a final device configuration shape of a non-woven tubular mesh. The different flow rates used resulted in different levels of burst release as shown in FIG. 19.

Though not wishing to be bound to any theory, it is believed that meshes utilizing these formulations demonstrate improved drug encapsulation characteristics (e.g. relative to the AC33 meshes described above) because the relatively large diameter of the fibers (>2 microns) can accommodate morphine sulphate particles having a cross-sectional dimension of approximately 2 microns formed by high-shear mixing processes.

### Example 7

### Control of Morphine Release by Selection of Sheath Polymer

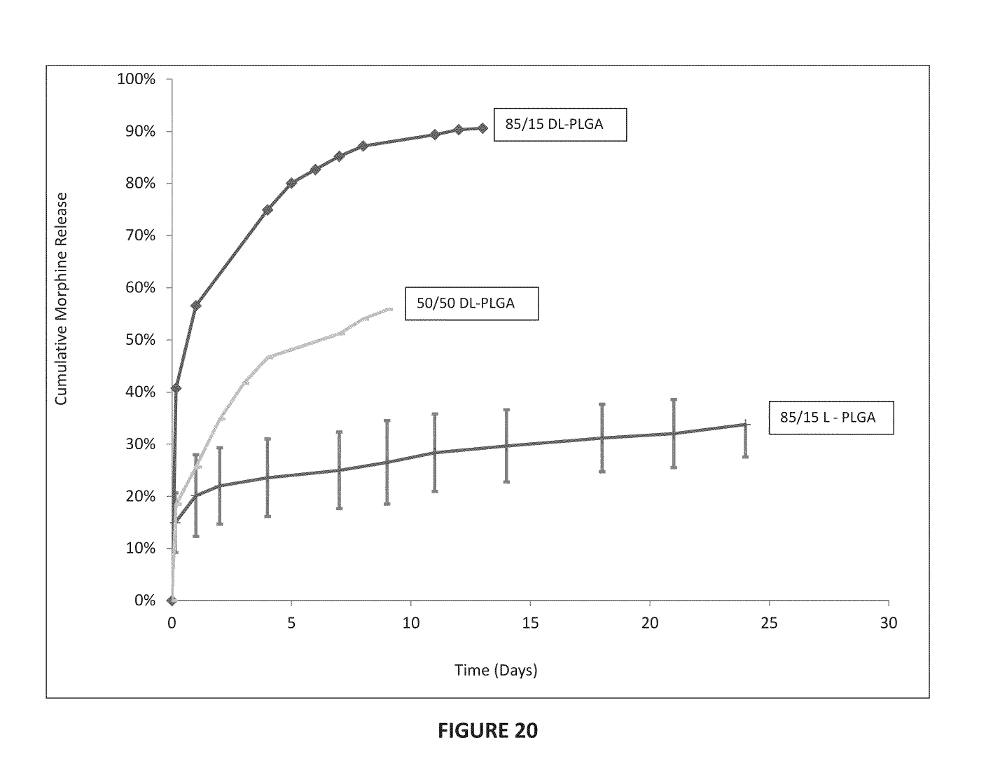

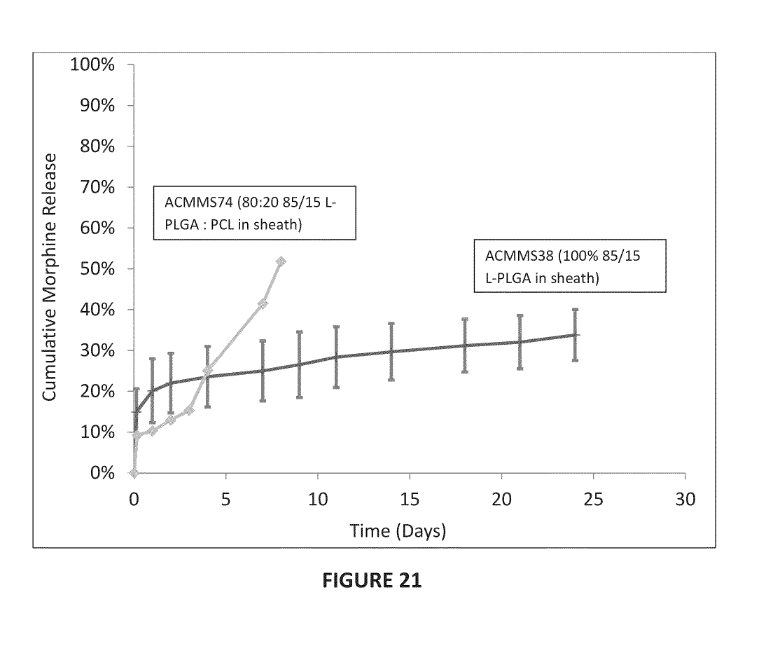

Release reates of morphine sulfate were influenced by the selection of sheath polymer. For example, instead of using 85/15 L-PLGA (as was used in ACMMS38), either 85/15 DL-PLGA or 50/50 DL-PLGA was used as the sheath polymer. All other fabrication conditions were kept the same. As can be seen in FIG. 20, different release profiles were achieved by changing the sheath composition. Release rates were also affected by the incorporation of PCL into the sheath polymer. We hypothesized that the addition of PCL into the sheath would enable drug to diffuse across it more easily, since morphine sulfate pentahydrate is completely or nearly completely released from PCL fibers in a rapid burst. ACMMS74 is a formulation in which we added 20% PCL relative to 85/15 L-PLGA in ACMMS38 formulation. We observed a faster daily release rate that occurred around day 3 (FIG. 21).

### Example 8

### Control of Morphine Release by Selection of Elution Medium

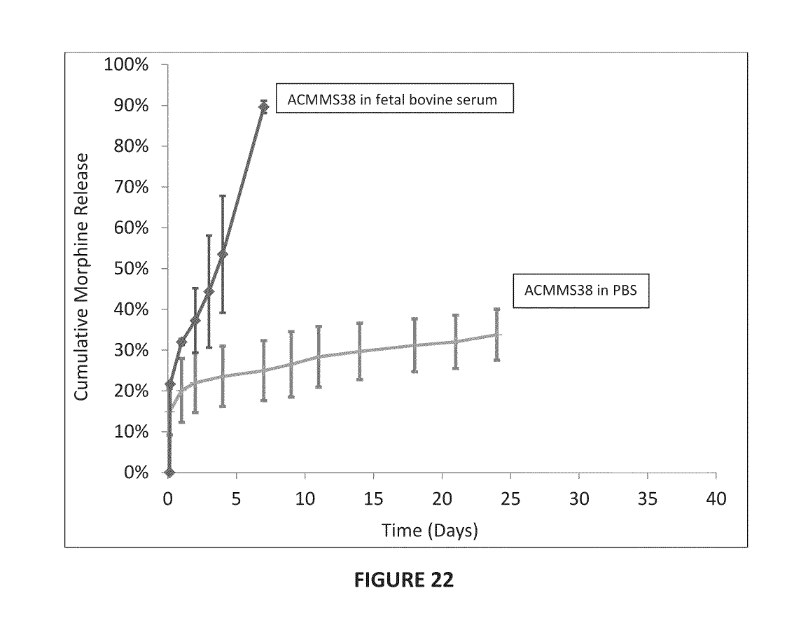

The inventors have also observed that the daily release of morphine sulfate can be impacted by the elution medium in which the sample is submerged in. We compared the elution of ACMMS38 in PBS vs. fetal bovine serum (diluted to a protein concentration of 11 g/L). The results indicated that a protein environment led to significantly faster release than in PBS (FIG. 22).

### Example 9

### Core-Sheath Fiber Meshes for Sustained Release of Morphine Base

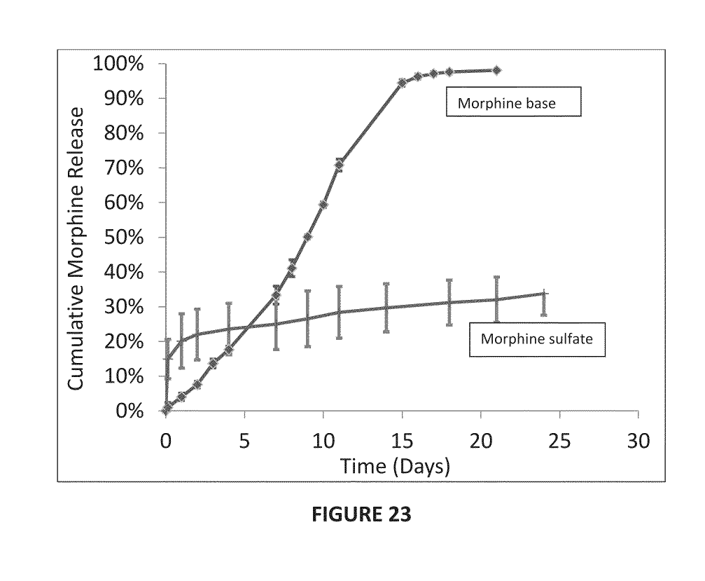

Formulation ACMMB1 is an electrospun mesh that contains morphine base instead of morphine sulfate. Fabrication of ACMMB1 occurs in a similar fashion as ACMMS38 except that the sheath solution is comprised of a 4.5% 85/15 PLGA in HFIP and the core solution is comprised of a 12 wt % PCL in HFIP containing 20% morphine base relative to the PCL. FIG. 23 illustrates the difference in elution profile in PBS at 37 C between the two formulations, demonstrating that the choice of drug or formulation impacts elution rate, and that closely-related formulations may have widely varying release kinetics when incorporated into implants of the invention.

### Example 10

### Improved loading of Morphine Sulphate in Core-Sheath Fibers

It has been observed during electrospinning that the flowability of the core solution decreases substantially when the morphine sulfate content is increased. For example, at 20% morphine sulfate, the core solution has flowability, can be pushed through a syringe, and subsequently be electrospun. However, at 40% morphine sulfate content, the solution no longer possesses any flowability (the solution exhibits a cream-like texture) that leads to difficulty in the formation of consistent core-sheath Taylor cones. The inability to load high amounts of drug into the core solution severely limits the total loading that can be achieved in resulting meshes. We have discovered that the flowability of morphine sulfate suspensions can be modulated by solvent choice. Specifically, by substituting the methanol component of the core solution in ACMMS38 for acetonitrile, we were able to incorporate more morphine sulfate while still maintaining good flowability (Table 11). For example, 40% morphine sulfate added to 15 wt % PCL in 6:1 (by vol) CHCl3:MeOH results in a cream-like suspension that has poor flowability; conversely, 40% morphine sulfate added to 15 wt % PCL in 6:1 (by vol) CHCl3:Acetonitrile still possessed good flowability.

TABLE 11
Impact of core solution solvent system on flowability and
relative drug loading

| | System A - 15 wt % | |
|---|---|---|
| | PCL in | System B - 15 wt % |
| | 6:1 (by vol) | PCL in 6:1 |
| Core Solution | CHCl3:MeOH | (by vol) CHCl3:ACN |
| Flowability at 20% | Good | Good |
| Morphine Sulfate Content | | |
| Flowability at 40% | Poor | Good |
| Morphine Sulfate Content | | |

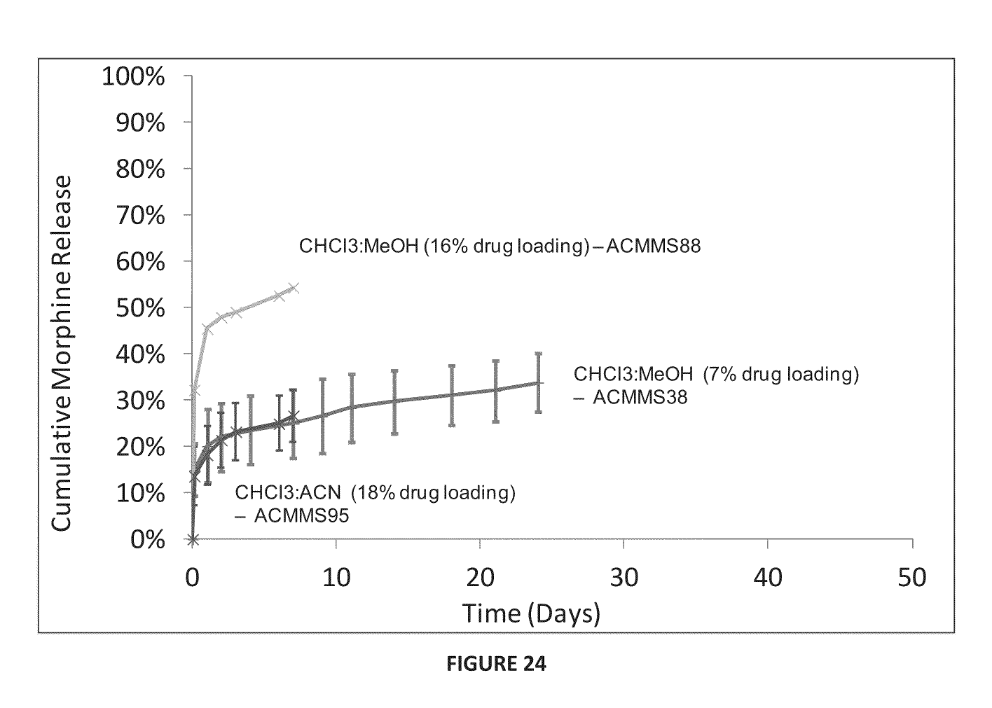

While not wishing to be bound to any theory, it is believed that acetonitrile has good wetting properties for morphine sulfate and therefore results in better dispersed morphine sulfate particles in solvent, leading to better flowability and/or hydrogen bonding with methanol leads to an increase in viscosity relative to acetonitrile. The ability to add 40% morphine sulfate into the core solution has a significant effect on the total drug loading. For example, the difference in the ability to incorporate 20% versus 40% drug into the core solution (and assuming everything else is equal) leads to an approximately two fold increase in total drug loading. FIG. 24 shows the cumulative release profile of ACMMS95, which uses system B with 40% morphine sulfate in the core; as shown, this formulation exhibits a low burst and subsequent sustained release even with 18% total drug loaded. Interestingly, the elution profile is very similar to that of ACMMS38, which only has a total drug loading of 7%. In general, higher loading formulations will exhibit a greater level of drug burst, as was observed in comparing formulation ACMMS38 with ACMMS88 (Both of these formulations used CHCl3:MeOH as the solvent in the core solution). We hypothesize that ACMMS95 is able to achieve a release profile similar to that of ACMMS38 at a higher loading due to the morphine sulfate having a more homogeneous drug particle distribution within the fiber (an effect from using ACN), resulting in less burst. Therefore, from a formulations perspective, in order to achieve core-sheath fibers with high drug loading and sustained release that exhibits low burst, it is desirable for drug to be well dispersed and the fibers large enough such that good encapsulation occurs.

### CONCLUSION

As used herein, the terms “drug” and “therapeutic agent” are used interchangeably to include small molecules, biologics, and other active ingredients used to produce a desired or expected biological effect. The term “threshold concentration” and the like is used herein to describe a concentration in tissue, serum, plasma, etc. at which such a certain biological effect is observed, such as a therapeutic effect or a side effect. Thus, a “therapeutic threshold concentration” or similar term may be used to refer to an ED₅₀, a dosing recommendation, or other effective concentration in the tissue of the patient. Similarly, The term “fiber” is used primarily to refer to electrospun, drug-loaded fibers as described in Palasis, and may include homogeneous fibers and core-sheath fibers as described in Palasis, as well as other drug-loaded fibers currently known or conceivable which may be assembled into higher-order structures such as yarns, ropes, tubes and patches. The invention is compatible with any such drug-loaded fibers.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

While various aspects and embodiments of the present invention have been described above, it should be understood that they have been presented by way of illustration rather than limitation. The breadth and scope of the present invention is intended to cover all modifications and variations that come within the scope of the following claims and their equivalents.

What is claimed is:

1. A method of treating a patient, comprising:
disposing, within a patient, an implant comprising a core-sheath fiber having a core comprising an analgesic and an outer diameter of no more than about 20 microns, wherein the core of the core-sheath fiber contains a first polymer and the sheath of the core-sheath fiber contains a second polymer different than the first polymer.

2. The method of claim 1, wherein the implant is one of a rope and a yarn, and disposing the implant within the tissue of a patient includes bending the implant to fit inside a space in or near the tissue of the patient.

3. The method of claim 1, wherein the implant is secured to a tissue of the patient by at least one of a tissue flap, a suture, a knot and a tissue fastener.

4. The method of claim 1, wherein the implant includes a coating that is erodible and/or biodegradable.

5. The method of claim 1, wherein the core of the core-sheath fiber contains a first polymer and the sheath of the core-sheath fiber contains a second polymer different than the first polymer.

6. A method of treating a patient, comprising:
disposing an implant within the patient to thereby increase a concentration of an analgesic in a tissue of the patient above a first threshold level for a period of at least one week, the implant including at least one core-sheath fiber having a core comprising the analgesic agent,
wherein a concentration of the analgesic within a plasma of the patient is not increased above a second threshold level for more than one day.

7. The method of claim 6, wherein the implant is disposed into a joint capsule and the tissue in which the concentration of analgesic is increased is a joint tissue.

8. The method of claim 7, wherein the implant is secured to a soft tissue in the joint capsule by a tissue flap.

9. The method of claim 7, wherein the implant is secured to a bone by a fastener.

10. The method of claim 7, wherein the implant is a rope or yarn and the implant is secured to a portion of the joint by at least one knot.

11. The method of claim 6, wherein the analgesic is released from the implant at a first rate over a period of approximately one day following implantation and, thereafter, at a second rate less than the first rate.

12. The method of claim 6, wherein the analgesic is released at a substantially constant rate until substantially all of the drug has been released from the implant.

13. A method of treating a patient, comprising:
during or after a surgical procedure on a joint of the patient, disposing an implant comprising a core-sheath fiber within the joint, the core-sheath fiber including a core comprising an analgesic,
wherein (a) the analgesic is released from the implant into the joint, thereby increasing a concentration of the analgesic within the joint above a first threshold value for a period of at least one week, and (b) a concentration of the analgesic does not exceed a second threshold value in a plasma of the patient for more than one day,
wherein the first threshold value is a concentration effective for relief or prevention of pain, and the second threshold is a concentration at which side effects are observed.

14. The method of claim 13, wherein the implant is disposed in at least one region of the joint selected from the group consisting of the lateral gutter, the medial gutter, the superior pouch, the suprapatellar space, the posterior lateral compartment and the posterior medial compartment.

15. The method of claim 13, wherein the analgesic is released from the implant over a period of at least one week.

16. The method of claim 13, wherein disposing the implant within the joint includes securing the implant by forming a knot therein.
